(19) United States
(12) Patent Application Publication
(10) Pub. No.: US 2025/0248660 A1
(43) Pub. Date: Aug. 7, 2025

(54) SYSTEM AND METHOD FOR CARDIOVASCULAR HEALTH ASSESSMENT AND RISK MANAGEMENT

(71) Applicant: 3P HEALTHCARE PTY LTD, Brisbane (AU)

(72) Inventor: Paul Beaver, Brisbane (AU)

(21) Appl. No.: 18/686,396

(22) PCT Filed: Aug. 24, 2022
(86) PCT No.: PCT/AU2022/050975

§ 371 Date: Feb. 23, 2024

(30) Foreign Application Priority Data
Aug. 26, 2021 (AU) 2021902785

(51) Int. Cl. A61B 5/00; A61B 5/021; A61B 5/024; G16H 10/40; G16H 10/60

(52) U.S. Cl. CPC A61B 5/7267; A61B 5/02108; A61B 5/02438; A61B 5/7275; G16H 10/40; G16H 10/60

(57) ABSTRACT

A cardiovascular health assessment model that identifies as well as stratifies individuals at risk of cardiovascular (CV) disease using low cost biomarkers more effectively than traditional CV models. The model allows for better management of ‘at risk’ or ‘high risk’ individuals by their medical/healthcare practitioner regarding the efficacy of nutrition, exercise, and lifestyle interventions. Individuals can take responsibility for their own health by having the ability to monitor their own health data, using new digital healthcare technologies connected to a bioinformatics platform, and thereby reduce the risk of a future adverse health event. The model enables practitioners to successfully integrate digital health and bioinformatics into their clinics to further improve the health and well-being of their patients, enhance the performance of their clinics, and ultimately reduce community healthcare costs.

### FIELD OF THE INVENTION

The present disclosure relates to improvements in systems and methods for identifying and managing physical outcomes related to degenerative cardiovascular health.

### BACKGROUND OF THE INVENTION

Globally healthcare systems around the world are in crisis because of an epidemic in chronic diseases such as obesity, Type 2 Diabetes (T2D) and cardiovascular disease (CVD), and the ageing population is only compounding the situation. As a result, healthcare systems are now being financially strained, and hence changing their focus from treating people when they become ill to seeking more personalised, preventative healthcare solutions.

Cardiovascular disease encompasses many different conditions, such as heart attacks, stroke and blood vessel diseases, and takes the life of one Australian every 12 minutes (45,600 people lost their lives to cardiovascular disease in 2011, being 31% of all deaths that year). It places a huge burden on our hospitals, for instance in 2009-10 there were 482,000 hospitalisations due to cardiovascular disease, it also played a secondary role in a further 800,000 hospitalisations. High blood pressure—also known as hypertension—is responsible for more deaths and disease worldwide than any other single health risk factor.

### 1. Traditional Risk Factors are not Closing the CVD Gap

The traditional cardiovascular disease (CVD) risk models which use the five cardiovascular risk factors, hypertension, dyslipidaemia, diabetes (impaired glucose tolerance), smoking and obesity, seem to have reached their limit as they can only identify approximately 50% of people, who will continue to have CV event. Furthermore, the ‘cholesterol centric’ models are not working as shown by research involving a large cohort of 136,905 patients from 541 hospitals admitted with Coronary Arterial Disease (CAD), where almost half had normal cholesterol levels.

The stiffness of our arteries is now recognised as a major risk factor for cardiovascular health diseases, such as hypertension. Extensive research has shown that three major factors responses of our blood vessels to at least 395 assaults are responsible for the stiffening of our arteries. These are: inflammation, oxidative stress (Free Radical Damage) and immune dysfunction.

2. Have not Identified New Biomarkers for these New Risk Factors

Biomarker parameters, such as central pulse pressure (PP) and heart rate (HR) can provide critical information about the health and wellbeing of a person. This infrequent collection of these measurements, as is the current practice, is problematic. Furthermore, low-cost anthropometric parameters, such as waist circumference (WC) or waist to height (Ht) ratio (WC/Ht), which give valuable data about stratifying adverse health outcomes, such as CVD and T2D are not commonly used in clinical practice.

Researchers are now looking at biomarkers associated with arterial elastic properties to use to stratify the risk of CVD and CHD, such as a variety of non-invasive vascular tests for endothelial dysfunction, arterial compliance, pulse wave velocity (PWV), augmentation index (AI), carotid intimal medial thickness (IMT), and coronary calcium score.

Pulse wave velocity (PWV) and augmentation index (AI) data generated by Central Aortic Pulse Wave Analysis (CAPWA) testing equipment, can help practitioners more effectively manage drug interventions. However, there does not appear to be much work done regarding using nutrition, exercise of lifestyle (NEL) interventions to reduce the CAPWA risk parameters apart from the standard ones of reducing BMI, stop smoking, reduce Cholesterol levels, etc.

### 3. Need a New CV Risk Model

The current Cardiovascular Risk Models are not working as evidenced by the fact that they can only identify 50% of individuals who have a heart attack. Therefore, new cardio-metabolic models are needed which include markers associated with the newly identified cardiovascular/metabolic biochemical processes, that is CV biomarkers.

Biomarkers could be used as a substitute or surrogate for a costlier clinical testing, such as pathology testing, or CT calcium score testing. Changes in these CV biomarkers could be detected earlier and at a lower cost to improve clinical outcomes, i.e., reduce adverse cardiovascular health outcomes and mortality.

### 4. Have not Embraced New Wearable Technologies

New technologies, such as mobile and wearable devices and digital diagnostics are starting to leverage the various streams of health data made possible by the ‘connected consumer’ to create powerful healthcare IT platforms that move beyond the current trendy apps and wearables, such as found in the fitness industry. Some tech savvy individuals are starting to take control of their health and wellbeing by monitoring health data using reliable mobile and wearable devices, and then wirelessly sending this health information in real time to secure IT platforms. These individuals, however, are in the minority although increasing in number.

5. Do not have a Connected Healthcare IT Platform

IT platforms which can connect individuals with their nominated clinicians, allied healthcare providers, caregivers and family members who can view the data and are alerted in times of concern, are now just starting to become available. Bioinformatics will be crucial to this change; however, it can be the bottleneck if the data cannot be collected, analysed, and presented in a ‘user friendly’ way. For example, the lack of ‘interoperability’ amongst health-related wearable devices currently limits big data's promise and, by extension, overall wellness and prevention initiatives. Interoperability has the potential to increase health outcomes, improve healthcare coordination and decrease costs.

More and more consumers are taking their health and wellness into their own hands; however, they are demanding Choice, Convenience and Control. This may be achieved primarily by
Unlocking, correlating, and interpreting information,
Empowering patients and practitioners,
Aggregating technologies,
Enabling real-time analytics,
Connecting all stakeholders, and
Bringing much-needed focus on personalised prevention.

### 6. Cardiovascular Health Assessment and Risk Management (CHARM) Model

The ability to accurately assess the cardiovascular health and then identify as well as stratify the risk of a patient is important for practitioners in their decision-making process as they must weigh up the benefits, risks and costs of the prevention strategies they choose for each patient. However, modelling using risk scores, whilst proving to be invaluable tools for identifying preventative interventions, have still not ‘closed the gap’ between the predicted and actual events. Reliance on traditional risk factors and not including more recent data regarding biochemical processes associated cardiovascular health can partly explain this gap.

New low-cost cardiovascular risk assessment models are needed which include markers associated with the newly identified cardiovascular biochemical processes, that is CV biomarkers. As defined by the American National Institute of Health, a biomarker is “a characteristic that is objectively measured and evaluated as an indicator of normal biological processes, pathogenic processes, or pharmacologic responses to a therapeutic intervention”.

Low-cost CV biomarkers could be used as a substitute or surrogate for a costlier clinical testing, such as pathology testing, or CT calcium score testing. Changes in these CV biomarkers could be detected earlier and at a lower cost to improve clinical outcomes, i.e., reduce adverse cardiovascular health outcomes and mortality.

One important cardiovascular biomarker in the CHARM model, which has received little recognition, is the subendocardial viability ratio (SEVR), which is an index of myocardial oxygen supply and demand, with low values representing poor myocardial perfusion. SEVR is an independent predictor of coronary flow in patients with essential hypertension. Furthermore, clinical research has shown that the SEVR is related to:
Central obesity and percentage body fat,
Type 2 Diabetes,
Arterial hardening in Type 2 Diabetic (T2D) patients,
Cardiovascular diseases,
Poor renal function,
All-cause mortality,
Cardiovascular biomarkers, such as central pulse pressure, heart rate and augmentation index (AIx),
Pathology markers associated with T2D, such as HbA1c, and with inflammation, such as IL-6, TNFα and CRP,
Poor physical fitness, and
Smoking.

An important feature of the CHARM model is the ability to establish a patient's SEVR value using low cost, readily available biomarkers without the need to use expensive medical testing equipment. Changes in these biomarkers can be detected earlier and at a lower cost to improve clinical outcomes i.e., reduce adverse cardiovascular health outcomes and mortality. Furthermore, these biomarkers are simple to monitor by the individual, and can be automatically uploaded to an individual's bio-informatics platform. For example, in addition, to knowing an individual's age, sex and height, the methodology in the patent, only needs the additional parameters of resting heart rate and waist circumference to be able to calculate the individual's SEVR value.

This methodology of calculating an individual's SEVR value has proven to have a very strong correlation (r=0.81 and above) with the SEVR values determined using the Pulse Wave Analysis (PWA) testing equipment, such as shown for example in the graphs of FIGS. 1 and 2.

The ability to accurately establish an individual's SEVR has a huge social relevance regarding community health as it allows for low cost scalability for large community groups, especially in remote areas without the need for expensive testing equipment.

Furthermore, there is a very strong correlation between the cardiovascular risk SEVR Biomarker, COVID-19 comorbidities, and Type 2 Diabetes, as shown in Table 1. This CHARM methodology may provide a very efficient, low cost and rapidly scalable method to establish the vulnerability of individuals with COVID-19 comorbidities, as well as stratify individuals infected regarding the severity of their response to this virus which has global implications in the fight against COVID-19. In addition, this model has applications for other chronic diseases where cardio-metabolic traits are comorbidities, such as Type 2 Diabetes.

TABLE 1

| | SEVR | COVID-19 | Type 2 |
|---|---|---|---|
| Physiological Trait | Biomarker¹ | Comorbidity | Diabetes |
| Hypertension | ✓ | ✓ | ✓ |
| Obesity | ✓ | ✓ | ✓ |
| Type-2 Diabetes | ✓ | ✓ | ✓ |
| Coronary Heart Disease | ✓ | ✓ | ✓ |
| Cardio-respiratory health | ✓ | ✓ | ✓ |
| Renal Function | ✓ | ✓ | ✓ |
| All-cause mortality | ✓ | ✓ | ✓ |
| Central haemodynamic markers | ✓ | —² | ✓ |
| Myocardial perfusion | ✓ | —² | ✓ |
| Inflammatory biomarkers IL-6, | ✓ | ✓ | ✓ |
| TNFα and CRP | | | |
| T2D pathology marker HbA1c | ✓ | ✓ | ✓ |

¹All physiological trait - CR biomarker associations, as for COVID-19 Comorbidities, are backed up by solid research in peer-reviewed scientific journals.
²Not yet established

The CHARM model also includes other novel low-cost cardiovascular health (CVH) surrogate biomarkers, in addition to the SEVR biomarker, based on the extensive evidence-based scientific research published in peer-reviewed journals and contained in the Knowledge database. These biomarkers are surrogates for clinical biomarkers associated with cardiorespiratory fitness (CRF), stiffness of both small and large arteries, inflammation, and percentage body fat. Preliminary in-clinic data from two medical centres has shown that the CHARM method has:

1. Been able to stratify a random group of patients on a gradient of risk that accurately identified the patients with established cardiovascular disease. This led to reassessment of the adequacy of their medical treatment and a review of diet, lifestyle and nutraceutical interventions.

2. Enabled the Practitioner to identify patients with a higher risk status and selectively focus more intensive medical and lifestyle interventions on some patients identified as at only intermediate risk by conventional assessments (such as Framingham risk scores).

3. A follow up visit some months later enabled the Medical Centre to document improved parameters of vascular function. It was interesting to note how readily this reflected improvement related to diet/exercise/nutraceutical interventions in those patients who had no further adjustments to their medications.

The CRF and CVH algorithms have been further evaluated over a 5-year period involving over 2,000 people in the original 2 medical clinics, as well as a pharmacy, a gymnasium, a manufacturing company, and individuals from the general community. This rigorous testing has shown that:
1. The calculated SEVR values have a very strong correlation with the values determined using the CAPWA medical testing equipment, and
2. This CVH assessment methodology is a very efficient, low cost and rapidly scalable method to establish both the CRF and CVH of individuals.

It will be clearly understood that, if a prior art publication is referred to herein, this reference does not constitute an admission that the publication forms part of the common general knowledge in the art in Australia or in any other country.

### SUMMARY

The present description in one preferred aspect provides for a system and method adapted to effectively identify cardiovascular conditions leading to severe health outcomes. Healthcare professionals, and others in the healthcare related industry may use elements of the system and method to more effectively identify, manage and treat patients, potentially saving many lives.

Presented is a model for improving cardiovascular risk assessment and enabling better identification of ‘at risk’ patients. The clinical application of the presently described model using evidence-based low cost CV biomarkers, identifies those patients at high risk of cardiovascular disease (CVD) as well as Type 2 Diabetes better than conventional methods.

This model, in one or more preferred aspects, uses a multi-disciplinary systematic approach incorporating systems biology, engineering and compliance risk management principles, physiology, genetics and metabolomics, behavioural psychology in a new personalised, preventative, and pro-active healthcare model for improving cardiovascular health individuals hence reducing community healthcare costs.

In particular, an advantage with this model, compared with conventional cardiovascular risk assessment models, is that it is based on a ‘Systems Biology’ approach and not just the traditional CV risk factors, such as hypertension, dyslipidaemia, diabetes (impaired glucose tolerance), smoking and obesity. A Systems Biology approach is a biology-based interdisciplinary methodology which focuses on connecting the molecular components, cells, tissues, and organs to physiological functions, based on biochemical pathways, which control our health and well-being. In other words, the objective of this unique approach is identifying the ‘biochemical drivers’ controlling our cardiovascular health, and when not functioning properly lead to adverse CV outcomes, that is, what are the ‘causes’ of poor CV health. In comparison, most traditional CV risk models are based on ‘associative’ research, i.e., dyslipidaemia, smoking, etc., without fully understanding the ‘causative’ nature of cardiovascular diseases.

This system and method is further enhanced by incorporating an individual's genetic profile, phenotypical data and health outcomes using a personalised, pro-active and preventative holistic approach. This system looks at gene to gene interactions in the major biochemical pathways which influence our cardiovascular health and well-being and no single gene SNPs in isolation. Consequently, this intervention can optimise cell health and gene expression to help the individuals improve their cardiovascular health.

In one or more preferred aspects, the present disclosure sets forth seven separate but connected stages.
1. An initial CV health assessment and risk management using low-cost biomarkers,
2. Pulse Wave Analysis testing,
3. Genetic Profiling,
5. Metabolomic testing,
6. Bioinformatics analysis,
7. Behavioural Psychology analysis, and
8. Use of Artificial Intelligence (AI) to optimise the CHARM model to allow scalability to community cohorts.

Each of these stages is populated with a variety of data from several different sources including anthropometric (i.e., body weight) measurements, blood pressure data, pulse wave analysis data, genetic data (preferably SNP data, and if relevant Copy Number Variation (CNV data), phenotype (i.e., lifestyle) data, traditional pathology and metabolomic data, as well as behavioural, lifestyle change and compliance data.

### 7. The Knowledge Database

An important aspect of this model is that it is backed up by extensive peer-reviewed scientific research from reputable scientific journals. An important consideration is an approach to understanding not only cardiovascular but the cardiorespiratory and cardiometabolic health of individuals, compared with traditional methodologies, as outlined previously. Since the scientific publications in these fields are expanding at an exponential rate, it is important that an up-to-data scientific knowledge database 119 (FIGS. 3-9) is available so that this approach is flexible, dynamic, and current.

The aspects of the scientific knowledge database include of the following:
1. The subject specific descriptors relevant for each Stage are used in web-based search engines for real time interrogation of on-line scientific databases containing peer-reviewed, evidence-based research articles, such as PubMed,
2. These articles are analysed, vetted by the scientific advisors and if relevant to the Systems Biology methodology, stored in the knowledge management database, and
3. This database can then be interrogated in each Stage as required using the subject specific descriptors relevant for that Module.

In one aspect, although not necessarily the only or the broadest aspect, a major advantage of the present invention resides in a method of determining an individual's cardiovascular health and then the most appropriate nutrition, exercise, and lifestyle (NEL) intervention strategy, based on the scientific evidence stored in the Knowledge database, the method including the steps of:
collecting subjective data from a subject;
collecting objective data from the subject;
receiving additional objective data concerning the subject;
determining a genetic ad metabolic profile of the subject;
evaluating the subjective data, the objective data, and the genetic profile of the subject; and
determining individual NEL intervention recommendations for the subject based on the subjective data, the objective data, and the genetic profile of the subject.

The method may also include evaluating the subjective data, the objective data and the genetic profile of the subject, and determining an individual using wearable devices, a bioinformatics platform, machine learning and artificial intelligence.

There are many advantages of associated with one or more aspects of the model described herein that arise from the seamless integration of these eight stages to empower both practitioners and individuals to improve health outcomes and reduce healthcare costs both for the individual and the community in general. In comparison, current healthcare models are in disparate and isolated silos with no interconnectivity.

### Cardiovascular Function—Definition

Cardiovascular function can be defined as relating to the circulatory system, which includes the heart and blood vessels and carries nutrients, glucose, and oxygen to the organs and tissues of the body and removes carbon dioxide and other wastes from the body. This broad definition includes, but not limited to, the following definitions:
Cardiorespiratory function, that is how the heart operates in partnership with the lungs to ensure the efficient transport of blood to and from the heart to facilitate the exchange of oxygen and carbon dioxide, and
Cardiometabolic function, that is how the heart and metabolic pathways (mainly glycemia and lipids) which involve several tissues (liver, fat, muscle, and others), function for, not only regulation of glycemia and lipids, but also in inflammation and hemostasias.

In addition, this includes the definitions involving the actual organs in the cardiovascular system, but not limited to the following:
Cardio function relating to the heart,
Vascular function, relating to the arteries, and
Endothelial function, relating to the ability of the endothelium to adequately perform its physiological roles, such as in the regulation of blood pressure and in hemostasias.

Abnormal cardiovascular function manifests as cardiovascular diseases which are conditions that affect the heart and blood vessels and include arteriosclerosis, coronary artery disease, heart valve disease, arrhythmia, heart failure, hypertension, orthostatic hypotension, shock, diseases of the aorta and its branches, disorders of the peripheral vascular system, and congenital heart disease.

The present description in one preferred aspect provides for a method for classifying cardiovascular function suspected of being abnormal in an individual, comprising: generating a set of features relating to data obtained about a subject which has the suspected abnormal issue, the data being derived from at least a pulse wave analysis, a DNA analysis, and an exercise and nutrition analysis; generating a feature vector for the cardiovascular function using the set of features; feeding the feature vector into a neural network with genetic algorithm for feature selection; and obtaining a result from the neural network relating to risk of cardiovascular disease.

In another preferred aspect, the present description provides a system for classifying cardiovascular function suspected of being abnormal in an individual. The system includes a pulse wave generator configured to measure cardiac efficiency. The system also includes a cardiovascular genomics database configured to retain data relating at least to glucose metabolism and cholesterol regulation, and a metabolomic database configured to retain data relating to at least genetic and environmental components of the individual. The system additionally includes a processor configured to utilise data from the pulse wave generator, cardiovascular genomics database, and metabolomic database to generate a cardiovascular health assessment of the individual.

In a further preferred aspect, the description provides for a system for classifying cardiovascular function suspected of being abnormal in an individual. The system includes a pulse wave generator configured to measure cardiac efficiency and output the data to be used to generate a set of features relating to the cardiovascular function of the individual. The system also includes a processor coupled to the pulse wave generator; the processor being configured to generate further features from at least one statistical calculation performed on the set of features. The system further includes a neural network configured to determine whether the cardiovascular function is abnormal utilising the set of features and the set of further features.

It is to be understood that both the foregoing general description and the following detailed description are exemplary and explanatory only and are not restrictive of the invention, as claimed. In the present specification and claims, the word “comprising” and its derivatives including “comprises” and “comprise” include each of the stated integers but does not exclude the inclusion of one or more further integers. It will be appreciated that reference herein to “preferred” or “preferably” is intended as exemplary only.

The claims as filed and attached with this specification are hereby incorporated by reference into the text of the present description.

The accompanying drawings, which are incorporated in and constitute a part of this specification, illustrate several embodiments of the invention and together with the description, serve to explain the principles of the invention.

### BRIEF DESCRIPTION OF THE FIGURES

FIG. 1 is a graph of actual versus predicted SEVR in healthy females.

FIG. 2 is a graph of actual versus predicted SEVR in healthy males.

FIG. 3 is a flow diagram of a stage 1 health pre-screening in accordance with a preferred embodiment of the present description.

FIG. 4 is a flow diagram of a stage 2 cardiovascular health assessment in accordance with a preferred embodiment of the present description

FIG. 5 is a flow diagram of a stage 3 factors and outcomes related to cardiovascular genomics in accordance with a preferred embodiment of the present description.

FIG. 6 is a flow diagram of a stage 4 factors and outcomes related to cardiovascular genomics metabolomic markers in accordance with a preferred embodiment of the present description.

FIG. 7 is a flow diagram of a stage 5 factors and outcomes related to a DNA reasoning engine in accordance with a preferred embodiment of the present description.

FIG. 8 is a flow diagram of a stage 6 factors and outcomes related to behaviour psychology in accordance with a preferred embodiment of the present description.

FIG. 9 is a flow diagram of how the CHARM model can be optimised for scalability to specific community cohorts, or vulnerable sub-cohorts for adverse cardiovascular health outcomes, such as the Australian, aborigines, the New Zealand Maoris, African Americans or the UK Black, Asian Ethnic Minorities (BAME).

FIG. 10 is a flow diagram of a method for classifying cardiovascular function suspected of being abnormal.

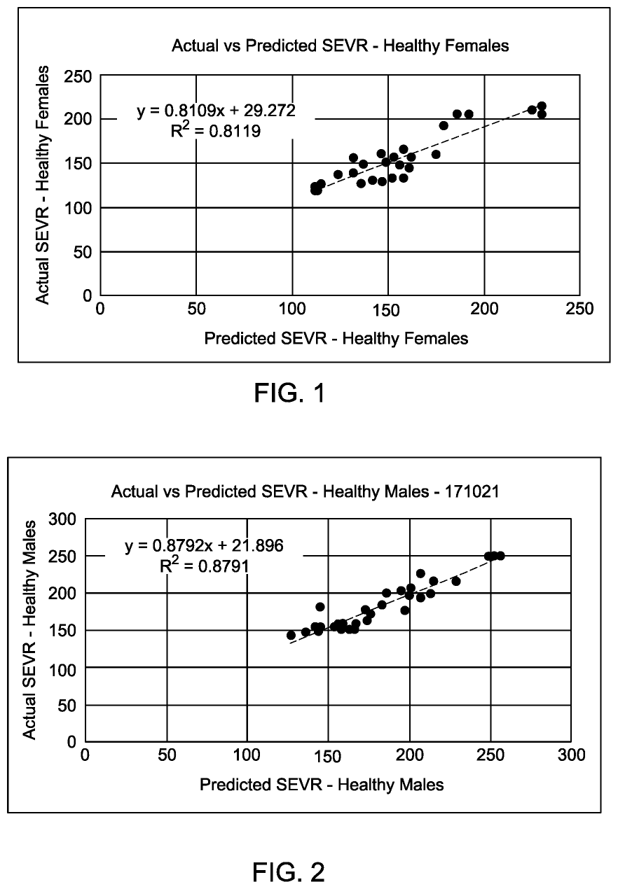

### DETAILED DESCRIPTION OF THE DRAWINGS

Reference will now be made in detail to the present preferred embodiments of the invention, examples of which are illustrated in the accompanying drawings.

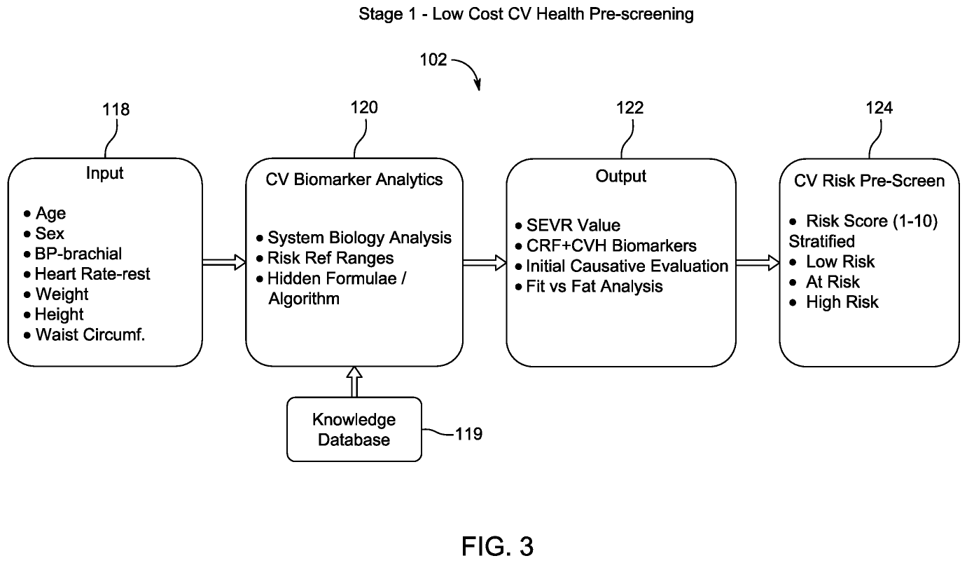

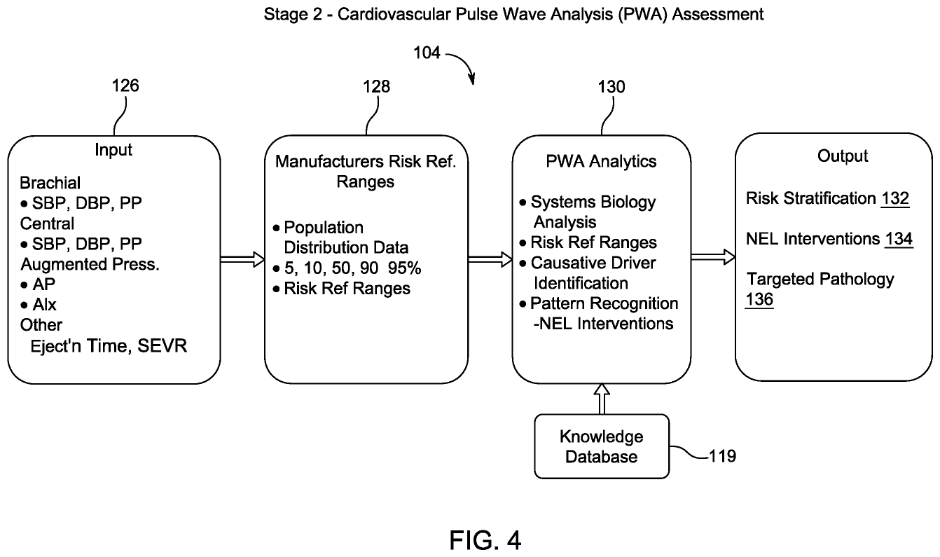

FIGS. 3 to 9 show a preferred embodiment of a seven-stage model 100 for cardiovascular health assessment and risk management. Model 100 includes generally seven stages, a pre-screening stage 102, a Computerised Arterial Pulse Wave Analysis (CAPWA) stage 104, a genomics stage 106, a Cardiovascular (CV) metabolomic marker analysis stage 108, a comprehensive bio-informatics data analysis stage 110, a behavioural analysis stage 112, and an optimising the CHARM model for scalability to community and other cohorts stage 114. The preferred elements of model 100 and their interrelationship are described further below.

### An Initial CV Health Assessment and Risk Management Using Low Cost Biomarkers.

Referring to FIG. 3, stage 1 shows the input of collective objective subject data based on the Systems Biology Analysis and evidence-based cardiovascular biomarkers 118. These low-cost biomarkers 118 may include, for example only, age, sex, resting heart rate, brachial blood pressure, waist circumference and height

A knowledge database 119 which contains evidence-based peer-reviewed scientific research regarding these physiological or biomarker parameters which can provide vital information regarding the cardiometabolic health and well-being of a person, many of which are not used in current clinical practice, is accessed in module 120 as part of the CV biomarker analytics As defined by the American National Institute of Health, a biomarker is “a characteristic that is objectively measured and evaluated as an indicator of normal biological processes, pathogenic processes, or pharmacologic responses to a therapeutic intervention”.

The CHARM methodology has identified biomarkers which may be used as a substitute or surrogate for a costlier clinical testing, such as pathology testing, or CT calcium score testing are included in database 119. Changes in these CV biomarkers could be detected earlier and at a lower cost to improve clinical outcomes, i.e., reduce adverse cardiovascular health outcomes and mortality. A surrogate endpoint is expected to predict clinical benefit/harm or lack thereof, based on epidemiologic, therapeutic, pathophysiological or other scientific evidence.

The biomarkers selected may be considered as a surrogate of cardiovascular events, a CV biomarker is chosen if they satisfy several criteria.
1. Proof of Concept
Do novel biomarker levels differ between subjects with and without outcome?
2. Prospective Validation
Does the novel biomarker predict development of future outcomes in a prospective cohort or nested case-cohort study?
3. Incremental Value
Does it add predictive information over and above established, standard risk markers?
4. Clinical Utility
Does it change predicted risk sufficiently to change recommended therapy?
5. Clinical Outcomes
Does the use of the novel biomarker improve clinical outcomes, especially when tested in a randomized clinical trial?
6. Cost-effectiveness
Does the use of the biomarker improve clinical outcomes sufficiently to justify the additional costs?
7. Ease of Use
Is it easy to use, allowing widespread application?
8. Methodological Consensus
Is the biomarker measured uniformly in different laboratories? Are study results directly comparable?
9. Reference values (or cut-off values)
Are there published reference values, or, at least, cut-off values?

Biomarkers 118 are analysed and interpreted using a methodology and criteria 120 to generate an output CV health assessment 122 and risk stratification score 124. This score can provide a general overview the individual's current cardiovascular health status, and by collecting subjective subject data of the individuals, current nutrition, exercise and lifestyle choices provide a preliminary overview of the efficacy of these choices.

### Pulse Wave Analysis Testing.

Referring to FIG. 4, Computerised Arterial Pulse Wave Analysis (CAPWA) stage 104 generates clinically recognised cardiovascular data and delivers an expedient, logistically efficient and affordable solution, allowing for remote and rapid stratification. Central aortic haemodynamic parameters, such as, but not limited to central Systolic and Diastolic Blood Pressure, Pulse Pressure, Augmented Pressure and amplification, or indexes that quantify pulse wave reflections, such as Augmentation Index (Aix), more accurately represent medical markers for cardiovascular risk, such as carotid intima-media thickness (CIMT) and left ventricular mass (LVM) than brachial blood pressure measurements.

The input for Stage 2 step 126 is obtained using a TGA/FDA approved CAPWA testing machine to capture at least the following information:
1. Study Date Time: the date and the time of the measurement;
2. Brachial Systolic Pressure: in mmHg;
3. Brachial Diastolic Pressure in mmHg;
4. Heart Rate: in bpm;
5. Central Systolic Pressure: in mmHg;
6. Central Diastolic Pressure: in mmHg;
7. Central Pulse Pressure: in mmHg;
8. Central Mean Pressure: in mmHg;
9. Central Augmentation Pressure: in mmHg;
10. Central Augmentation Index: in %;
11. Ejection Duration in ms;
12. Buckberg SEVR: Sub-Endocardial Viability Ratio in %;
13. Ejection Duration in %; and
14. Aortic Aix (P2/P1) in %.

Several clinical validation studies for the CAPWA testing equipment have been undertaken. For example, in one study the CAPWA results over 4,000 patients were validated against the parameters determined from invasive CV testing. In addition, large population studies have shown that the CAPWA parameters can predict the occurrence of heart failure, CV events and CV mortality. Research for a variety of different cohorts has shown that this technology is clinically more useful with better clinical outcomes and is more cost effective than invasive CV testing. Risks identified through studies such as those mentioned above may be used to generate a risk profile 128 with classifications of ‘low risk’, ‘at risk, and ‘high risk’ based on a normal population distribution data analysis, such as 5, 10, 50, 90 and 95%.

Reference ranges for the central haemodynamic parameters have been published and meet the criteria to be classified as clinical surrogate CV endpoints or biomarkers. This data resides in Knowledge Database 119.

In addition, the knowledge database 119 contains evidence-based peer-reviewed scientific research regarding central haemodynamic indexes, measured using the Central Aortic Pulse Wave Analysis (CAPWA), associated with the potential risk of future clinical CV events including:
1. Central aortic workload,
2. Arterial resistance both peripheral and central,
3. Cardiorespiratory efficiency—oxygen supply and demand,
4. Fit versus fat paradox,
5. Heart rate variability—atrial fibrillation, arrythmia etc., and
6. Heart failure paradox.

Knowledge database 119 also preferably contains scientific research regarding the physiological drivers and biochemical pathways associated with these central haemodynamic indexes, the risk reference ranges plus the associations with the biomarkers mentioned previously.

At part of the CAPWA testing and analysis protocol at least the following personal data will be collected for each subject
1. Gender
2. Date of Birth
3. Age
4. Weight: in kilograms
5. Height: In centimetres
6. Body Mass Index: In kg/cm2
7. Waist Circumference: in centimetres
8. A smoker: (yes/no)
9. Family history of CVD: (yes/no)
10. On any CV/Diabetic medication: (yes/no).

The PWA analysis 130 is based on the Systems Biology approach, using data from Knowledge Database to interrogate and identify the physiological drivers and biochemical pathways associated with:
1. Central aortic workload,
2. Arterial resistance both peripheral and central,
3. Cardiorespiratory efficiency—oxygen supply and demand,
4. Fit versus fat paradox,
5. Heart rate variability—atrial fibrillation, arrythmia etc, and
6. Heart failure paradox.

Causative drivers are identified, stratified, 132 and then targeted nutrition, exercise, and lifestyle interventions output 134, are generated, and uploaded to the bioinformatics platform. The CHARM model is then able to track improvements in CV health based on the recommended nutrition, exercise, and lifestyle interventions output in 134.

### CV Related Pathology Markers

A vast array of circulating biomarkers related to different aspects of CV pathophysiology has been proposed as candidates for refinement of CV risk prediction. The present patent focuses on functional pathology biomarkers that are related to vascular wall biology, blood flow haemodynamics, inflammation, coagulation, cholesterol regulation, 136. For example, C-reactive protein (CRP) is a circulating biomarker related to vascular wall biology with a large body of published studies supporting its clinical use for risk stratification. Other circulating pathology biomarkers include, but are not limited to:
Homocysteine,
Vitamin D—25(OH)D,
D-dimer
HbA1c, and
HDL/LDL/TG.

### Genetic Profiling and Phenotype Analysis.

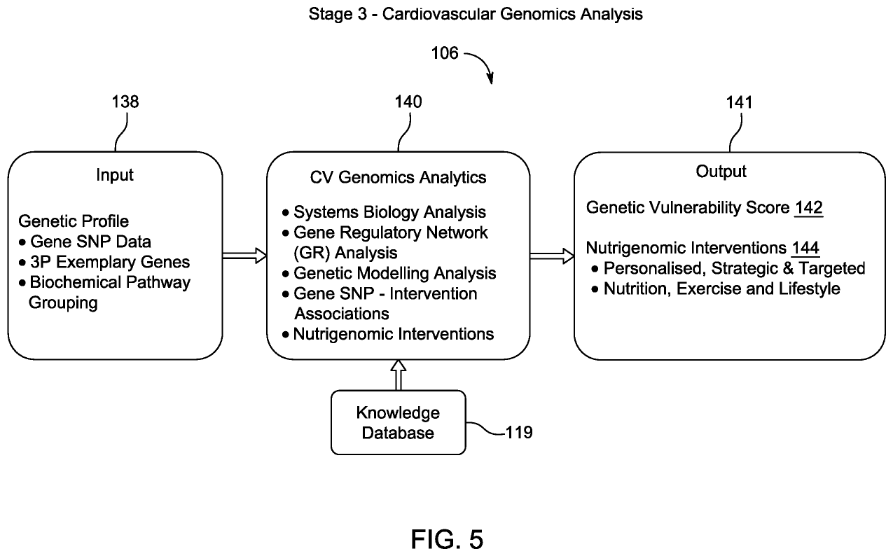

Referring now to FIG. 5, Stage 3 Genomics can include genetics, nutrigenetics, nutrigenomics, metabolomics, and proteomics. The main difference between genomics and genetics is that genetics scrutinises the functioning and composition of the single gene whereas genomics addresses all genes and their inter-relationships in order to identify their combined influence on the growth and development of the organism. In addition, Nutrigenetics examines how our body responds to nutrients, exercise and lifestyle based on our inherited genes, whereas Nutrigenomics can be defined as the interaction between our genes and our nutrition, exercise and lifestyle (NEL) choices. In essence, Nutrigenomics impacts our health status and hence disease risk susceptibility, and potentially alter our nutritional requirements.

The genotype analysis Stage 3, 106, is directed toward the determination of the target individual's personal genetic profile from a biological sample, such as, for example, a saliva sample or a mouth swab (for cheek epidermal cells). A genetic test profile panel of multiple genes which influence cardiovascular and metabolic health is generated based on the advanced Systems Biology—biochemical pathway-gene regulatory network (GRN) analysis. Exemplary genes in the critical biochemical pathways impacting cardiovascular health are analysed at step 138 after being identified using Systems Biology, biochemical pathway, Cardiovascular, and PWA related genes using the evidence-based research data in Knowledge Database 119 to generate a Cardiovascular Health (CV) Genetic Profile.

Exemplary genes selected for this nutrigenetic profile 138 include the genes that involve in the following biological functions or pathways, but are not limited to:
1. Vascular Health and matrix regulation,
2. Endothelial function,
3. Blood pressure regulation,
4. Haemodynamics of blood flow,
5. Coagulation and Fibrinoysis,
6. Inflammation and immune response,
7. Redox regulation and cytoprotection,
8. Methylation and homocysteine regulation,
9. Fat metabolism, and
10. Glucose metabolism and insulin regulation.

The exemplary genes in this CV Genetic Profile that are being analysed for genetic variants, known as Single Nucleotide Polymorphisms (SNPs), in the above selected biochemical pathways which influence our CV health include, but are not limited to:

Adiponectin, ADIPOQ, Angiotensin Converting Enzyme, ACE, Angiotensin II Receptor-1, AGTR1, Angiotensinogen Gene, AGT, Apopliprotein A1 APOA1, Apopliprotein B, APOB, Cholesteryl Ester Transfer Protein, CETP, Coagulation Factor F1, F11, Coagulation Factor F13, F13, Coagulation Factor F2, F2, Coagulation Factor F5, F5, Coagulation Factor F7, F7, Coagulation Factor F9, F9, Collagen Type I Alpha 1, COL1A1, Collagen Type I Alpha 5, COL1A5, C-reactive Protein, CRP Cyclooxygenase-2, COX-2, Cystathionine-&beta; -synthase, CBS, Elastin, ELN, Endothelial Nitric Oxide Synthase, eNOS3, Endothelin Receptor Type A, EDNRA, Fat Mass and Obesity Gene, FTO, Fatty Acid Binding Protein 2, FABP2, Fatty Acid Desaturase, FADS1, Glucokinase Regulatory Protein, GCKR, Glucokinase, GCK, Glucose 6 Phosphatase Catalytic subunit, G6PC2, Glutathione S-transferase P1, GSTP1, G-Protein &beta;3 subunit, GNB3 Heme Oxygenase-1, NQ01, Hepatic lipase, LIPC, Insulin-Like Growth Factor, IGF2BP2, Interleukin-1, IL-10, Interleukin-18, IL-18, Interleukin-1β, IL-1-β, Interleukin-6 Receptor, IL-6R, Interleukin-6, IL-6, Interleukin-8, IL-8, Leptin Receptor, LEPR, Low-Density-Lipoprotein Receptor, LDLR, Matrix Metalloproteinase-3, MMP-3, Matrix Metalloproteinase-9, MMP-9, Methionine Synthase Reductase, MTRR, Methionine Synthase, MTR, Methylenetetrafydrofolate Dehydrogenase, Methylenetetrafydrofolate Cyclohydrolase, Formyltetrahydrofolate Synthetase, MTHFD1, Methylenetetrahydrofolate Reductase, MTHFR, Monocyte Chemoattractant Protein-1, MCP-1, NADP-Oxidase p22 phox, NADPH-CYBA, Paraoxonase-1, PON-1, Peroxisome Proliferator-Activated-&gamma, PPARγ, Plasminogen Activator Inhibitor-1, PAI-1, Potassium inward. rect. channel, subfam. J-11, KCNJ11, Quinone Reductase-NADS-(P) Sex Hormone-Binding Globulin, SHBG Transcription factor 7-like 2, TCF7L2, Tumour Necrosis Factor Alpha, TNFα, Vascular Endothelial Growth Factor, VEGFa, Vitamin D Receptor, VDR, Zinc Transporter Protein Member 8, SLC30A8.

While a number of means of assessing a target individual's genetic profile are available, a preferred method includes the following steps: a target individual provides a sample of their buccal cells using a DNA self-collection kit; the DNA sample is sent to an accredited DNA testing laboratory for analysis; the laboratory generates a unique genetic profile using specified exemplary cardiovascular genes determined in this invention, taking into account nominated SNP variants; the test results are verified and assessed for quality control by a molecular geneticist; the verified test results are analysed and presented in a personal genetic profile report. In addition, a genetic cardiovascular vulnerability score can be determined.

The Cardiovascular Genomic Analysis 140 looks at gene to gene interactions in the major biochemical pathways which influence our cardiovascular health and well-being and no single gene SNPs in isolation, i.e., a gene regulatory network (GRN) analysis. The purpose of this analysis is to optimise cell health and gene expression to help the individuals improve their cardiovascular health.

This stage also includes a personalised, subject-specific nutrition, exercise and lifestyle intervention recommendations based on subject subjective and objective data, as well as an individual's genetic profile. The Phenotype (gene-lifestyle-environment interaction) analysis focuses on the target individual's current nutrition, exercise and lifestyle choices using data regarding the target individual's current health status. These inputs are designed to help identify any potential symptoms that indicate that the target individual's body is out-of-balance with regard to various physiological functions, such as, for example, inflammation at the cellular level, circulation problems and high levels of homocysteine which influence cardiovascular health.

Data inputs may relate to, or be directed to, a variety of topics relevant to providing an individual health intervention strategy, and include, for example, enquiries concerning attitude, nutrition and diet, lifestyle, exercise, habits, preferences, work, stress, hobbies, personal health, personal/family health history, fitness, and goals.

The data generated from the enquiries can be analysed to assess the effects of the target individual's current nutrition, exercise, and lifestyle choices on physiological functions in the body, such as cellular health. In addition, this analysis can help identify if the target individual is showing any adverse symptoms resulting from these effects at the cellular level. The data collected for this module can be used to establish the target individual's current phenotype.

In addition to the collection of data regarding the target individual's current nutrition, exercise and lifestyle choices using the various enquiries, the collection of objective data from the target individual can be realised via anthropometric measurements, pathology markers and/or the completion of one or more biometric tests, as described herein, to further populate the data in this stage.

This embodiment relates to a personalised, subject-specific nutrition, exercise and lifestyle intervention recommendations based on subject subjective and objective data, as well as an individual's genetic profile.

A cardiovascular genetic vulnerability/risk scoring methodology 140, based on the evidence-based research in Knowledge Database 119, using a Compliance Risk Management process of Likelihood, Exposure and Consequences (LEC) has been developed. The likelihood for each gene SNP, in each biochemical pathway is determined by the frequency of the risk allele in any population or sub-population.

Exposure is determined by the subject's current nutrition, exercise and lifestyle choices, and environment. For example, if a subject has a variant in the ACE, AGT, or GNB3 genes and they consume more than 1.5 gms of sodium chloride (common salt) then their risk of a cardiovascular event is increased as a result of increased blood pressure (hypertension).

The consequences are determined by the results of genetic association studies, in the Knowledge Database which links the evidence of a correlation between the trait, i.e., biological function, such as endothelial function, or disease state, such as Cardiovascular Disease (CVD) and the specific gene SNP. The statistical relationship between the specific SNP genotype and the CV trait is determined from the evidenced based research by either:
1. OR (odds ratio), used in case-controlled studies, which is the odds of one genotype occurring, i.e., the risk genotype, relative to the odds of the other genotype occurring, or
2. RR (relative risk), used in cohort studies and randomised clinical trials, which is the probability that a person with a particular genotype will have that trait or develop that disease relative to the probability of someone without that genotype.

The actual genetic vulnerability score (GVS) for each genotype or SNP, is determined by the relevant genetic models, whether the Dominant or Recessive Model. Dominance of one of the alleles can be assumed by treating the heterozygote and the homozygote variant risk genotype as a single category. For example, if the alleles of the gene of interest are A and B in haploid, and B is the ‘increasing’ or ‘risk’ allele, i.e., the one causing an adverse trait or effect, the three genotype groups would then be AA, AB and BB.

From a physiological point of view this this can be described as follows,
AA—both genes in the pair contribute to the normal functioning of the gene product,
AB—one of the genes in the pair results in a compromised functioning of the gene product, and
BB—both genes in the pair results in a more compromised functioning of the gene product.

This dichotomization of the SNP genotypes can be determined by comparing the OR and or RR values for each of the genotypes and classified as follows:
Dominant: ‘BB+AB’ versus ‘AA’, and
Recessive: ‘BB’ versus ‘AB+AA’.

The choice of which model to use is determined by the data in the Knowledge Database and the appropriate score classification is chosen as follows in Table 2 below.

TABLE 2
Genetic Vulnerability Scores for each
Genotype and Genetic Risk Model

| | AA | AB | BB |
|---|---|---|---|
| Recessive | 0 | 1 | 3 |
| Dominant | 0 | 2 | 3 |

Once the relevant genetic model and associated scoring system are identified for each gene variant, then a total GVS 142 can be generated for each of the 10 CV related biochemical pathways listed in 138: the higher the score, the greater the potential impact on the subjects CV health as a result of the variants in that CV related pathway. This genetic vulnerability scoring methodology allows for a CV causative risk stratification of the various biochemical pathways and hence, allows for more strategic, personalised, and targeted interventions to improve the CV health of a specific subject.

Use of machine learning and Artificial Intelligence (AI) for the DNA reasoning engine will enhance this Genetic Vulnerability Score methodology and optimise the actual scores allocated in Table 1, based on continuous servo-loop feedback and pattern recognition as the subject database increases for both populations and sub-populations, i.e., Americans—African Americans, Hispanics, Latinos, etc. Furthermore, optimisation will occur as the evidence-based research contained in Knowledge Database #3 is continually updated, expanded and analysed.

As stated above, Nutrigenomics can be defined as the interaction between human genes and human nutrition, exercise, and lifestyle (NEL) choices, and hence impacts our health status, disease risk susceptibility, and potentially alter human nutritional requirements. For example, research has shown that a daily dose of 3 to 5 gms of combined DHA and EPA in a ratio of 3 parts EPA to 2 parts DHA can reduce blood pressure by reducing the impact of pro-inflammatory cytokines, such as, IL-1B, IL-6, TNFα and CRP. Furthermore, the effectiveness of supplementing with EPA and DHA is dose dependent, as using less than 2 gm of EPA+DHA, whilst having a beneficial nutritional effect, generally fails to show any Nutrigenomic effect.

Once the GVSs have been established in 140, they are compared and cross-checked with output data from 122, 132, and analysed for associations between the genetic vulnerabilities and compromised functioning of the particular trait. Output from this analysis, will then determine the most appropriate personalised and strategic Nutrigenomic intervention 144 (nutrition, exercise and lifestyle intervention program) for that subject.

### Metabolomic Analysis.

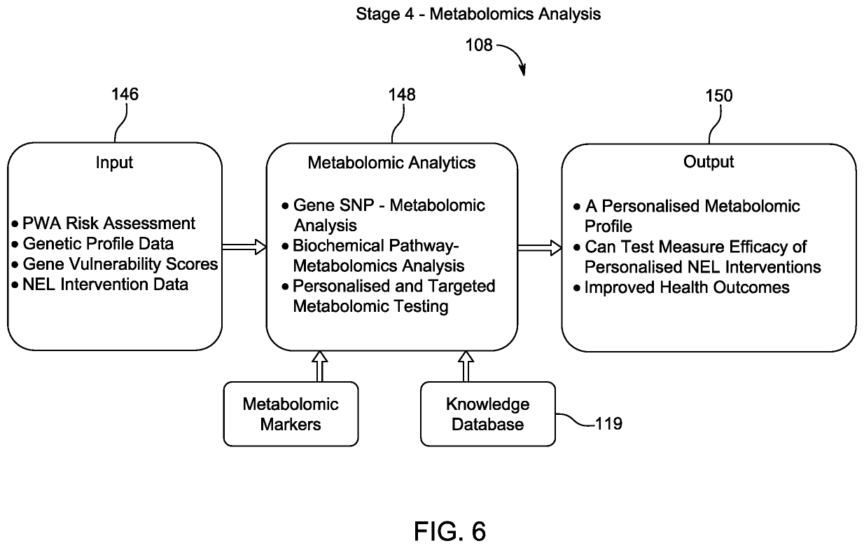

Referring now to FIG. 6, Stage 4, Metabolomics can be defined as the screening of small-molecule metabolites present in samples of biological origins. The analysis of certain metabolites (‘metabolome’) can provide an index, of biomarkers, of a current biological state of an individual, that is provide a snapshot of the current cardiovascular and metabolic health of an individual. By comparing metabolomic profiles, patterns of variations between different groups can be determined: healthy versus diseased, as well as between individuals with different genetic vulnerabilities regarding cardiovascular health. In addition, metabolomics can be used to monitor the outcome of nutrition, exercise, and lifestyle interventions as part of the CHARM model.

Unlike an individual's genetic profile, which remains static, their metabolomic profile reflects the effects of both genetic and environmental components, or nutrigenomic effects, including nutrition, exercise and lifestyle choice, environmental factor as well as medications, such as cholesterol lowering drugs.

Thus, a strategic cardiovascular metabolomic profile can offer a level of description of the cardiovascular biological system that transcends pure genetic information and more closely reflects the ultimate phenotypes. The analysis of genetic profiling and metabolomics is an important part of this system's biological approach to improving an individual's cardiovascular health and well-being. For example, Interleukin 6 (IL-6) is a circulating biomarker related to vascular wall biology. Other circulating biomarkers, including oxidized low-density lipoprotein and dysfunctional high-density lipoprotein, are well-placed for prevention.

This embodiment identifies certain metabolomic markers based on the individual's genotype, using the cardiovascular genetic profile mentioned in Stage 3 (FIG. 5) and include, but not limited to:
1) INFLAMMATION
   a) Pro-inflammatory Cytokines
      Interleukin-1 (IL-1), Interleukin-6 (IL-6), Interleukin-8 (IL-8), Interleukin-18 (IL-18), Tumour necrosis factor alpha (TNFα), C-reactive Protein (hs-CRP), Leptin (LEP), glycoprotein acetylation (GlycA),
      Arachidonic acid (Ω6): eicosapentaenoic acid ratio (Ω3) (AA:EPA Ratio),
   b) Anti-inflammatory Cytokines
      Interleukin-10 (IL-10);
2) ENDOGENOUS DEFENCE—Redox Regulation and Cytoprotection
   a) Oxidative Stress
   8-hydroxy-2′-deoxyguanosine, (8OHdG), Malondialdehyde (MDA), Advanced glycated end products receptor (RAGE),
   b) Cytoprotection
   Glutathione (GSH), Ratio Reduced and oxidised state of Glutathione (GSH:GSSH);
3) CARDIOVASCULAR HEALTH
   a) Vascular Endothelial Health
   Plasminogen Activator Inhibitor-1 (PAI-1), Adiponectin (ADIPOQ), Cell adhesion molecule CAM,
   b) Blood Pressure Regulation
   Angiotensin I (AGTI), Angiotensin II (AGTII), Angiotensin-converting enzyme (ACE),
   c) Blood Coagulation
   Fibrinogen, D-dimer,
   d) Methylation
   Homocysteine (Hcy).
4) VITAMIN D PATHWAY
   Vitamin D (25(OH)D), Vitamin D (1,25(OH)2D).
5) FAT METABOLISM and CHOLESTEROL REGULATION
   a) Lipid-related markers
   Oxidised Cholesterol-Low Density Lipoproteins (oxLDL), Apolipoprotein A and B (ApoA and ApoB), Apolipoprotein A (ApoA), Lipoprotein (a); and
6) GLUCOSE METABOLISM and INSULIN REGULATION
   a) Biomarkers of glycemia
   Glycated haemoglobin (HbA1c).

As shown in FIG. 6, for Stage 4, a CV metabolomic marker analysis 108 allows one or more scientific advisors to maintain and update the various intervention templates used to analyse the subjective data, objective data and genetic data collected/received concerning the target individual, as well as to determine the appropriate individual health recommendation for the target individual.

Maintenance of the various templates can be done at any time and can also be done jointly or in isolation of each other.

### Bio-Informatics Analysis.

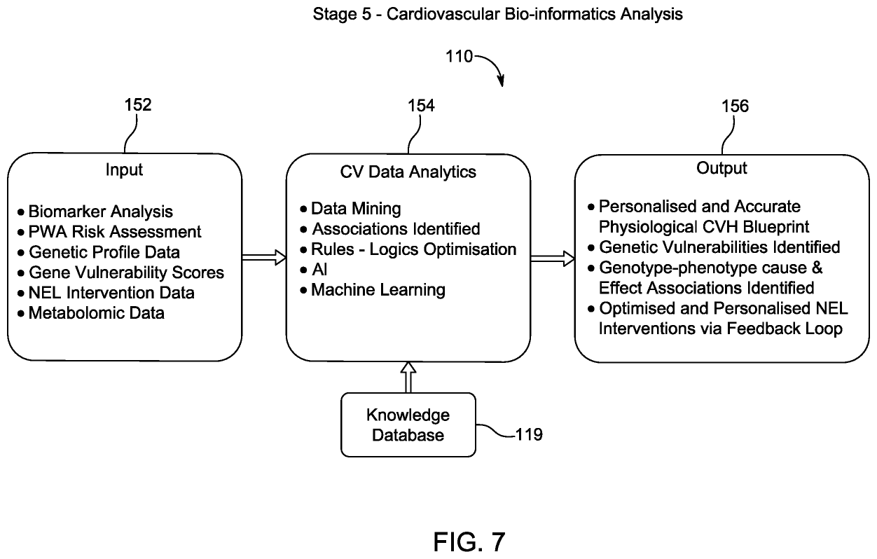

In Stage 5, FIG. 7, the output 150 (FIG. 6) and the outputs 122, 132,134, 136, and 141 from all the other stages are brought together for a bio-informatics analysis to generate a unique and personalised physiological/biochemical cardiovascular function blueprint for each individual, an individual health recommendation for the target individual. In addition, this analysis can identify whether the cause of any adverse cardiovascular health outcomes is a result of potential genetic vulnerabilities, or poor nutrition, exercise or lifestyle choices.

The gene variations for the target individual are analysed against the genetic research data in the knowledge database, to determine the intervention(s) they should apply, based on their genetic analysis and the identified potential genetic vulnerabilities. Determination of the appropriate intervention(s) is also affected by the target individual's subjective and objective data, as interpreted via the appropriate nutrition, exercise and lifestyle templates.

The output 156 of this stage is an individual's health recommendation for the target individual, including one or more appropriate interventions, along with their individual weightings. The individual health recommendation can take the form of a personalised report, that includes one or more nutrient, exercise and lifestyle interventions for the target individual. It can also include one or more focus areas for the target individual, specifically directing the attention of the target individual to areas of nutrition, exercise and/or lifestyle that are a high priority for addressing.

### Behavioural Psychology Analysis.

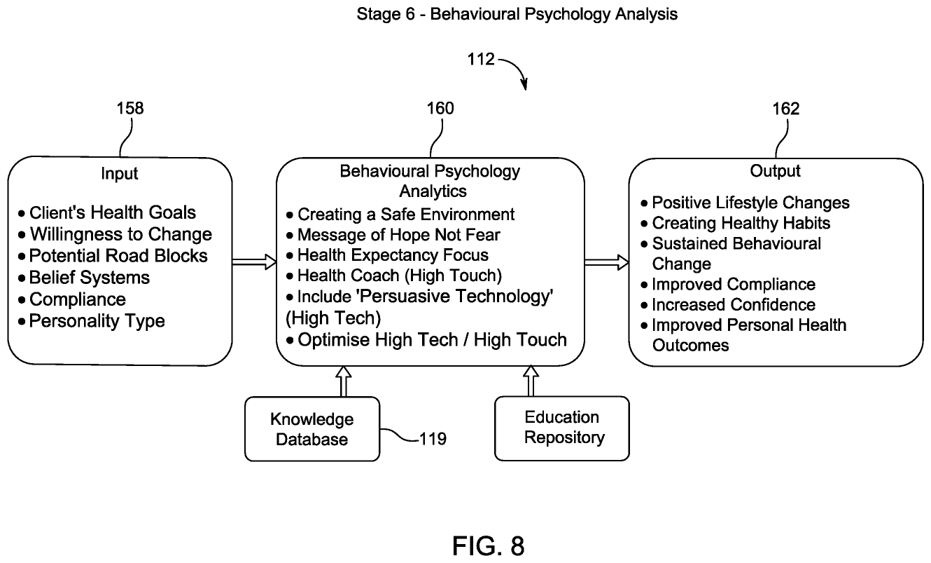

Referring now to FIG. 8, Stage 6 in order to achieve success with an individual's health intervention strategy, it is necessary to determine if the target individual is serious about wanting to make the necessary nutrition, exercise and lifestyle changes to improve their overall health and wellbeing.

A significant decrease in the adherence to healthy nutrition, exercise and lifestyle (NEL) habits, such as eating 5 serves of fruit and vegetables per day, exercise for 30 minutes 4 times per week or moderate alcohol consumption has been associated with the global increase in chronic lifestyle diseases such as hypertension, cardiovascular diseases (CVD) and Type 2 Diabetes (T2D). Behavioural psychology research involving medical practitioners and their patients has identified why their patients will not do what is good for them? When asked this question by their practitioner there is generally only two types of answers:
1. ‘I know I should, I know it would be good for me’ BUT ‘I'm not ready to change’, and
2. I don't want a lecture (that is why they withhold information from their Practitioner).

Furthermore, research has shown that there are two components of motivation for behaviour change:
1. A perception of a need to change, and a cost/benefit analysis base on core values important to the patient, and
2. The confidence and belief in the ability to change.
(If people do not believe they can succeed to change then they are unlikely to try seeing any effort as futile and hence lose confidence.)

Research has shown that personal genetic profiling significantly improved motivation and compliance in patients. For example:
‘Genetic profile/risk information, such as for Type 2 Diabetes, might have greater value to motivate behavioural change compared with standard health risk information’.
‘Patient feedback from genetic testing for obesity risk showed that higher-risk results had positive motivational effects with minimal changes in negative effect from fatalism’.
‘Genetic testing has the potential to be a useful clinical or preventative tool when combined with appropriate information.’

Specific input inquiries 158 are used to collect subjective data from the target individuals, to capture information to include, but not limited to the following:
1. Health goals,
2. Attitude,
3. Level of confidence in achieving their health goal,
4. Willingness/readiness to change,
5. Perceived roadblocks to change,
6. Current belief systems, and
7. Personality types.

In module 160, the focus is on creating an environment with a focus on health expectancy not life expectancy, hope not fear, and tailored to their personality. Persuasive technology, i.e., technology that is designed to change attitudes or behaviours of the users through persuasion and social influence, but not through coercion, can be incorporated into the individual's bio-informatics platform.

This information can be captured using the aforementioned enquiries and can be used to assess the target individual's commitment to making the necessary nutrition, exercise and lifestyle changes to improve their overall health and wellbeing.

Assuming that a target individual is ready to make the necessary nutrition, exercise and lifestyle changes to improve their overall health and wellbeing, additional data collected in the enquiries can be used to establish their personal goals and their level of confidence in achieving those goals. Information gathered for this module will also help to identify the level of support that the target individual will require from their health coach, personal trainer, consultant, or other qualified individual, and if they will need further additional life coaching input.

A final aspect of this module is a determination by the target individual of an area of focus that matches their goals, and can include, for example, as well as cardiovascular health, weight management, women's health, men's health, anti-ageing maintenance of cognitive function, and combinations thereof.

Individuals can have access to a health coach and or nominated healthcare practitioner by their practitioner portal and determine the ratio of high tech/high touch their required to make and the sustain the necessary behavioural change.

The outputs 162 of Stage 6 include the following:
a. Sustainable behavioural change,
b. Improved compliance,
c. Increased confidence,
d. Achieving health goals, and
e. Improved cardiovascular health.

### Use of Artificial Intelligence (AI) to Optimise the CHARM Model to Allow Scalability to Community Cohorts.

The randomized placebo-controlled clinical trial (RCT) has achieved iconic status in the field of medical research. For many decades these trials have represented a scientific ‘gold standard’ in which medical practitioners and specialists invest both their trust and confidence. The RCT model is, indeed, a useful tool for a variety of reasons, however, it is neither perfect nor infallible. A significant amount of clinical trial data has very limited therapeutic success (from 1-in-4 to 1-in-25 patients).

To a large extent, the poor translation of RCT data to patient outcome experience is due to the fact that chronic diseases such as cardiovascular disease, all have multiple triggering (causative) factors determined by the unique way that a person's genes interact with lifestyle, diet, and environment. While symptoms related to these diseases are common in terms of presentation, the triggering events that initiate the onset of disease in each individual are highly variable.

An underlying major problem with RCTs is that they are inherently based on a “one size fits all” approach, trials that invariably need to be large in size to demonstrate a reproducibility and to show evidence of effectiveness. Since it is now recognized that there is significant biological heterogeneity within any specific disease diagnostic group, the new science of Precision Medicine has evolved where studies focus on a single person-known as N-of-1 trials. Consequently, there is a need to apply new approaches that integrate developments in biometrics, bioinformatics, and N-of-1 trial design into criteria that measure evidence of effectiveness. This approach could be described as moving from population-based data to that of individualised responses.

If enough n-of-1 data are collected over a sufficiently long period of time, and control interventions are used, such as in input 164, then the participating individuals can be identified as responding or not responding to the intervention. Functional assessment in combination with new biometrics and bioinformatics tools represents a powerful step forward in the development of innovative approaches to collecting and documenting evidence in support of patient specific interventions. Consequently, the aggregation of the results for many N-of-1 trials such as for analytics module 168, will offer valuable cohort or sub-cohort databases for improving community health and thereby reduce healthcare costs.

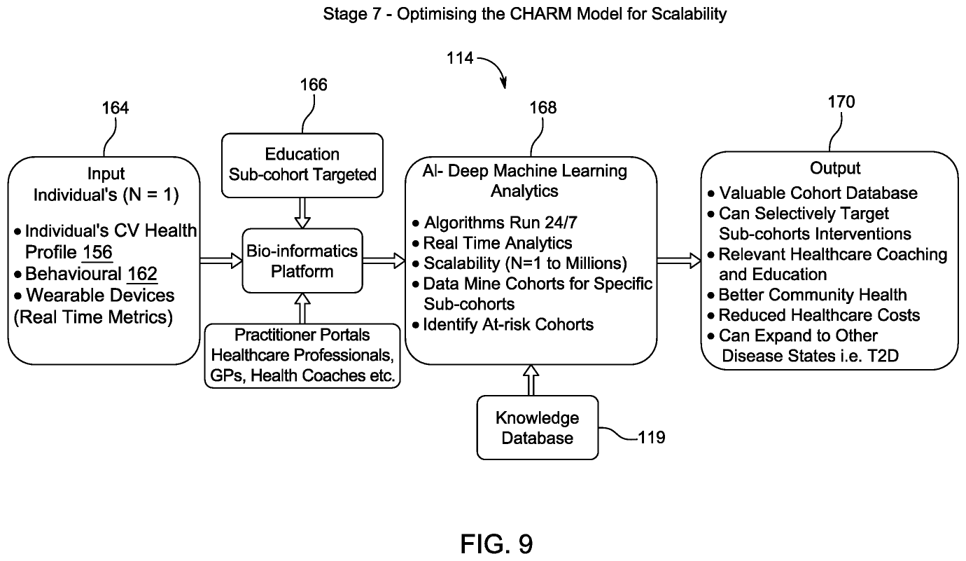

Referring to FIG. 9 for Stage 7, outputs from output 156 (FIG. 7), personalised physiological/biochemical cardiovascular function blueprint, 162, the behavioural profile and real time metrics from wearable devices help make up input 164 for an individual's bio-informatics platform 166.

Combining the outputs from input 164 with practitioner portals, access as determined by the individual, via individual bio-informatic platforms 166 allows for improving individual health outcomes, identifying genetic vulnerabilities and phenotype contributions, stratifying the major drivers influencing the individual's CV health. Outputs from platforms 166 for the N-of-1 can be combined with the N-of-1 for selected cohorts or sub-cohorts in AI module 168 will allow for scalability and the ability to generate valuable community-based cohort data, identify and selectively target at risk sub-cohorts, identify the most appropriate healthcare coaching support, tailor the most relevant education and consequently generate positive changes in community health to help reduce national healthcare cost.

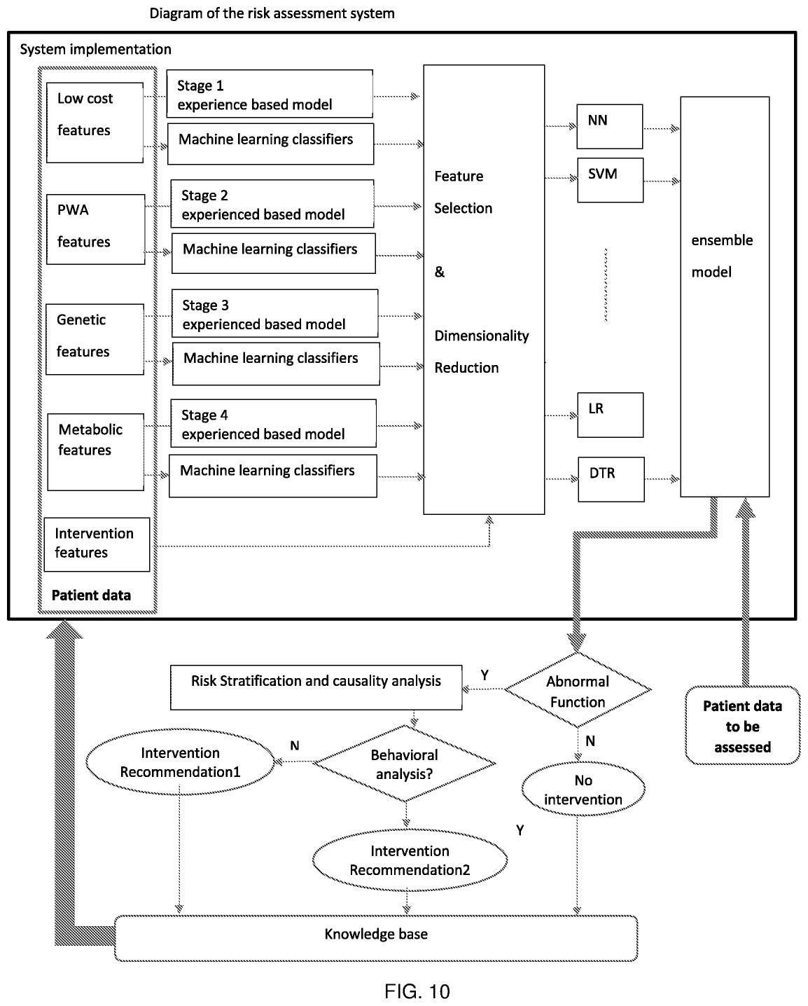

Referring to FIG. 10, the system 100 is implemented with knowledge base 119 which includes the data extracted from the literatures and the data from individual patients, using statistical and machine learning methods in combination with our experienced based models. Patient data is classified though system 100 using a variety of classification models, and an output generated to determine cardiovascular risk. Based on the risk level and further patient behavioural analysis, a recommendation of intervention is given. Each assessment record will be added to the knowledge base and be used for keeping the system up to date.

Having described the preferred components of system 100, a preferred method for classifying cardiovascular function suspected of being abnormal in an individual will now be described with reference to FIGS. 3-9. First, a set of features relating to data obtained about a subject which has the suspected abnormal cardiovascular function is generated. The data is derived from at least a pulse wave analysis, a DNA analysis, and an exercise and nutrition analysis. Next, a few feature vectors for the cardiovascular function assessment are generated based on the categories of the whole set of features including low cost biomarker features, PWA derived features, genetic or metabolic features. The feature vectors are fed into a set of classifiers including the experience-based model and statistical-based classifiers (first level classifiers). The output from these classifiers is fed to the next level machine learning-based classification models (second level classifiers) in conjunction with a genetic algorithm for feature selection as a preferred feature selection algorithm. These machine learning based classification models are preferably a neural network (NN), support vector machine (SVM), logistic regression (LR) and decision tree (DTR). An ensemble model is built based on the output from each classification model based on a weighted majority voting method (can be substituted with other ensemble modelling methods).

The set of features is preferably generated using biomarkers, including at least one of age, gender, resting heart rate, waist circumference, and height of the individual. It will be appreciated that other biomarkers could be used in lieu of, or in addition to those listed above. The set of features is preferably generated using data derived from a metabolomic analysis. The metabolomic analysis is preferably derived from both genetic and environmental components.

Once a set of features has been generated, the set is preferably categorized into low-cost features, PWA features, genetic features and metabolic features. Each group of the features is classified using one or more classifier models, preferably the first level classifiers. There are a variety of techniques suitable for use as a classifier. Suitable classifiers include, but are not limited to statistical applications (e.g., Bayesian, K-nearest neighbour, fuzzy pyramid linking, discriminant analysis (DA), logistic regression (LR), multivariant adaptive regression splines (MARS), support vector machine (SVM), and Hidden Markov Model), neural networks (parallel, double, deep learning recurrent), decision trees, random forest, associated rule mining, and case-based reasoning, or a combination of any of the foregoing.

The features are preferably normalised before the feature file can be efficiently used by the classifiers. Thus, the method further includes the step of normalising the features prior to feeding the first level classifiers. The method further preferably includes performing a classification model using the sets of categorized features to generate the feature vectors. In one or more preferred embodiments, the classification model is Support Vector Machine, and/or a Bayesian classifier. The set of features is preferably generated using biomarkers, including at least a cardiorespiratory fitness parameter and/or subendocardial viability ratio. The cardiorespiratory fitness (CRF) refers to the ability of the circulatory and respiratory systems to supply oxygen to skeletal muscles during sustained physical activity. CRF parameters include VO2max and subendocardial viability ratio (SEVR). The method further preferably includes obtaining a result from the neural network relating to a risk of diabetes of the individual.

The second level classifiers are preferably one or more of neural network, support vector machine, logistic regression and decision tree. The input for the second level classifiers is preferably selected by genetic algorithm from the combination of the output of the set of first level classifiers, the experience-based models and the normalised sets of features.

It will be appreciated that the steps described above may be performed in a different order, varied, or omitted entirely without departing from the scope of the present invention.

The foregoing description is by way of example only and may be varied considerably without departing from the scope of the present invention. For example, although cardiovascular “function” is mentioned above, it will be appreciated that this may include abnormal functioning of arterial walls and other structures associated with the pulmonary and circulatory system. Cardiovascular disease risk is a condition certainly determinable with elements of the present model, but aspects of the model and methods may be adapted for conditions or chronic diseases such as, but not limited to metabolic disorders like Type 2 diabetes and immune disorders like SARS and COVID 19.

For artificial intelligence applications, other applications may be used in place of, or in addition to above mentioned classifiers for a final ensemble model. For example, random forest may be used to substitute decision tree, and a K-nearest neighbour-based model can be added for building the final ensemble model. For feature selection and dimensionality reduction to feed second level classifiers, other algorithms maybe used instead of a genetic algorithm, for example Principle Component Analysis (PCA). The PCA output can be used to feed the second level classifiers. For another example, stepwise features selection can be also used.

The features described with respect to one embodiment may be applied to other embodiments or combined with or interchanged with the features of other embodiments, as appropriate, without departing from the scope of the present invention.

The present description in a preferred form provides the advantages that are often lacking in known systems or models, such as:
A new Cardiovascular Health Assessment and Risk Management (CHARM) model for improving cardiovascular risk assessment and enabling better identification of ‘at risk’ patients,
Is based on Systems Biology approach which has identified the ‘biochemical drivers’ controlling our cardiovascular health, that is, what are the ‘causes’ of poor CV health compared with most traditional CV risk models which are based on ‘associative’ research,
It augments existing medical science,
New low-cost CV biomarkers have been identified which can be detected earlier to improve clinical outcomes, i.e., reduce adverse cardiovascular health outcomes and mortality,
The model is easily scalable to CV risk stratify large cohorts/populations and sub-populations where hypertension and CVD are comorbidities, i.e., COVID-19,
The model incorporates an individual's genetic profile, phenotypical data and health outcomes using a personalised, pro-active and preventative holistic approach to identify the most appropriate personalised and strategic nutrition, exercise and lifestyle (NEL) intervention strategy, and
Empowers both practitioners and individuals to improve health outcomes and reduce healthcare costs both for the individual and the community in general.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for classifying cardiovascular function suspected of being abnormal in an individual, comprising:
generating a set of features relating to data obtained about a subject which has the suspected abnormal issue, the data being derived from at least a subendocardial viability ratio (SEVR) determination obtained from a pulse wave analysis, a DNA analysis, and an exercise and nutrition analysis;
generating sets of feature vectors for the cardiovascular function using the set of features related to different type of data;
feeding all sets of features into a classification model;
selecting features using a genetic algorithm to feed the classification model;
obtaining an individual result from multiple classification models separately; and
obtaining a result from an ensemble model relating to risk of cardiovascular disease from abnormal cardiovascular function.

2. The method of claim 1, wherein the classifier models include at least one of logistic regression, neural network, support vector machine, decision tree, random forest and an experienced based model.

3. The method of claim 1, wherein the ensemble model is derived from a combination of at least two different classification models.

4. The method of claim 1, wherein the SEVR determination utilises at least one of age, gender, resting heart rate, waist circumference, brachial blood pressure, weight, and height of the individual.

5. The method of claim 1, wherein a set of features is generated using data derived from a metabolomic analysis.

6. The method of claim 1, wherein a set of features is generated using data derived from a genomic analysis, the genomic analysis being derived from both genetic and environmental components.

7. The method of claim 1, further comprising normalising the selection features prior to feeding the classifiers.

8. The method of claim 1, further comprising performing a statistical or machine learning models using the set of features categorised by data type to generate further features to be added to the whole set of existing features.

9. The method of claim 1, wherein the classification model is a logistic regression.

10. The method of claim 1, wherein the classification model is support vector machine.

11. The method of claim 1, wherein the classification model is a Bayesian classifier.

12. The method of claim 1, wherein the classification model is a mixed model.

13. The method of claim 1, wherein the set of features is generated using biomarkers, including at least a cardiorespiratory fitness parameter.

14. The method of claim 1, further comprising obtaining a result from the neural network relating to risk of diabetes of the individual.

15. A system for classifying cardiovascular function suspected of being abnormal in an individual, comprising:
a pulse wave generator configured to measure cardiac efficiency;
a cardiovascular genomics database configured to retain data relating at least to glucose metabolism and cholesterol regulation;
a metabolomic database configured to retain data relating to at least genetic and environmental components of the individual; and
a processor configured to utilise data from the pulse wave generator, cardiovascular genomics database, and metabolomic database to generate a cardiovascular health assessment of the individual.

16. The system of claim 15, wherein said processor is configured to determine abnormality of the cardiovascular function using data transmitted from a wearable health monitoring device.

17. The system of claim 16, wherein the data transmitted from the health monitoring device includes heartrate.

18. The system of claim 16, wherein the data transmitted from the health monitoring device includes resting heartrate.

19. A system for classifying cardiovascular function suspected of being abnormal in an individual, comprising:
a pulse wave generator configured to measure cardiac efficiency data to be used to generate a set of selection features relating to the cardiovascular function of the individual;
a processor coupled to said pulse wave generator, said processor being configured to generate further section features from at least one statistical calculation performed on said set of section features; and
a neural network configured to determine whether the cardiovascular function is abnormal utilising the set of section features and the set of further selection features.

20-21. (canceled)

22. The system of claim 19, wherein said processor is configured to generate further selection features from at least one statistical calculation performed on data derived from a metabolomic database having genetic and environmental components relating to the individual.
